(19) United States
(12) Patent Application Publication
(10) Pub. No.: US 2005/0065158 A1
(43) Pub. Date: Mar. 24, 2005

(54) Treatment of sexual dysfunction

(75) Inventors: Alasdair Mark Naylor, Kent (GB); Pieter Hadewijn Van Der Graaf, Kent (GB); Christopher Peter Wayman, Kent (GB)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/883,622

(22) Filed: Jul. 1, 2004

Related U.S. Application Data
(60) Provisional application No. 60/512,030, filed on Oct. 17, 2003.
(60) Provisional application No. 60/513,125, filed on Oct. 21, 2003.

(30) Foreign Application Priority Data
Jul. 16, 2003 (GB) 0316673.3
Aug. 1, 2003 (GB) 0318095.7
Sep. 11, 2003 (GB) 0321308.9

(51) Int. Cl. A61K 31/519

(52) U.S. Cl. 514/252.16; 514/262.1

(57) ABSTRACT

This invention relates to the use of cyclic guanosine 3′, 5′-monophosphate phosphodiesterase type five (PDE5) inhibitors, in combination with 5HT1a agonists for the treatment of sexual dysfunction, particularly female sexual arousal disorder (FSAD) with concomitant hypoactive sexual desire disorder (HSDD).

### CROSS-REFERENCE TO RELATED APPLICATION

This application claims priority from United Kingdom Application Number 0316673.3, filed on Jul. 16, 2003, United Kingdom Application Number 0318095.7, filed on Aug. 1, 2003, United Kingdom Application Number 0321308.9, filed on Sep. 11, 2003 and the benefit from U.S. Provisional Application No. 60/512,030, filed on Oct. 17, 2003 and U.S. Provisional Application No. 60/513,125, filed on Oct. 21, 2003.

This invention relates to the use of cyclic guanosine 3′, 5′-monophosphate phosphodiesterase type five (PDE5) inhibitors, in combination with 5HT1a agonists for the treatment of sexual dysfunction, particularly female sexual arousal disorder (FSAD) with concomitant hypoactive sexual desire disorder (HSDD).

Male sexual dysfunction includes male erectile dysfunction, ejaculatory disorders such as premature ejaculation (PE), anorgasmia (inability to achieve orgasm) and desire disorders such as hypoactive sexual desire disorder (lack of interest in sex).

The categories of female sexual dysfunction (FSD) are best defined by contrasting them to the phases of normal female sexual response: desire, arousal and orgasm (see S R Leiblum, (1998), Definition and Classification of Female Sexual Disorders, Int. J. Impotence Res., 10, S104-S106). Desire or libido is the drive for sexual expression. Its manifestations often include sexual thoughts either when in the company of an interested partner or when exposed to other erotic stimuli. Arousal includes the vascular response to sexual stimulation, an important component of which is genital engorgement and increased vaginal lubrication, elongation of the vagina and increased genital sensation/sensitivity and a subjective excitement response. Orgasm is the release of sexual tension that has culminated during arousal. Hence, FSD occurs when a woman has an absent, inadequate or unsatisfactory response in any one or more of these phases, usually desire, arousal or orgasm.

The American Psychiatric Association classifies female sexual dysfunction (FSD) into four classes: FSAD, hypoactive sexual desire disorder (HSDD), female orgasmic disorder (FOD), and sexual pain disorders (e.g. dyspareunia and vaginismus) [see the American Psychiatric Association's Diagnostic and Statistical Manual of Mental Disorders, 4th Edition (DSM-IV)].

DSM-IV defines the four classes as follows:
HSDD—Persistently or recurrently deficient (or absent) sexual fantasies and desire for sexual activity which causes marked distress or interpersonal difficulties. The judgement of deficiency or absence is made by the clinician, taking into account factors that affect functioning, such as age and the context of the persons life.
FSAD—Persistent or recurrent inability to attain, or to maintain until completion of the sexual activity, an adequate lubrication-swelling response of sexual excitement.
FOD—Persistent or recurrent delay in, or absence of, orgasm following a normal sexual excitement phase. Women exhibit wide variability in the type or intensity of stimulation that triggers orgasm. The diagnosis of FOD should be based on the clinician's judgement that the woman's orgasmic capacity is less than would be reasonable for her age, sexual experience, and the adequacy of the sexual stimulation she receives.
Sexual Pain Disorders such as Dyspareunia and Vaginismus. Dyspareunia is the recurrent or persistent genital pain associated with sexual intercourse. Vaginismus is the recurrent or persistent involuntary spasm of the musculature of the outer third of the vagina that interferes with sexual intercourse.

More recently the American Foundation for Urologic Disease has developed definitions using the same four classes (see The Journal of Urology, 2000, Vol 163, page 888-893). The definitions are along similar lines as follows to DSM-IV:
HSDD is the persistent or recurrent deficiency (or absence) of sexual fantasies/thoughts, and/or desire for or receptivity to sexual activity, which causes personal distress.
FSAD is the persistent or recurrent inability to attain or maintain sufficient sexual excitement, causing personal distress, which may be expressed as a lack of subjective excitement, or genital (lubrication/swelling) or other somatic responses.
FOD is the persistent or recurrent difficulty, delay in or absence of attaining orgasm following sufficient sexual stimulation and arousal, which causes personal distress.
Sexual pain disorders: Dyspareunia is the recurrent or persistent genital pain associated with sexual intercourse. Vaginismus is the recurrent or persistent involuntary spasm of the musculature of the outer third of the vagina that interferes with vaginal penetration, which causes personal distress.

HSDD is present if a woman has no or little desire to be sexual, and has no or few sexual thoughts or fantasies. This type of FSD can be caused by low testosterone levels, due either to natural menopause or to surgical menopause. Other causes in both pre-menopausal woman (i.e. woman who are pre-menopausal and who have not have hysterectomies) as well as post menopausal women include illness, medications, fatigue, depression and/or anxiety. Factors having a potential (conscious or sub-conscious) psychological impact such as relationship difficulties or religious factors may be related to the presence of/development of HSDD in females.

FSAD is a highly prevalent sexual disorder affecting pre-, peri-, and post menopausal women. It is associated with concomitant disorders such as depression, cardiovascular diseases, diabetes and UG disorders. FSAD is characterised by inadequate genital response to sexual stimulation. The genitalia do not undergo the engorgement that characterises normal sexual arousal. The vaginal walls are poorly lubricated, so that intercourse is painful. Orgasms may be impeded. FSAD can be caused by reduced oestrogen at menopause or after childbirth and during lactation, as well as by illnesses, with vascular components such as diabetes and atherosclerosis. Other causes result from treatment with diuretics, antihistamines, antidepressants e.g. selective serotonin reuptake inhibitors or antihypertensive agents.

Sexual pain disorders (includes dyspareunia and vaginismus) are characterised by pain resulting from penetration and sexual activity and may be caused by medications which reduce lubrication, endometriosis, pelvic inflammatory disease, inflammatory bowel disease or urinary tract problems.

We have now found that PDE5 inhibitors in combination with 5HT1a agonists work well in treating subjects who suffer from sexual dysfunction. The combination may be deemed synergistic.

Suitable sexual dysfunctions include FSAD, HSDD and FOD in women and MED in men.

We have found that PDE5 inhibitors in combination with 5HT1a agonists work particularly well in FSAD subjects who suffer from concurrent significant HSDD. The combination may be deemed synergistic.

According to a first aspect, the invention provides the use of a PDE5 inhibitor in combination with a 5HT1a agonist in the manufacture of a medicament for the treatment of sexual dysfunction

According to a preferred aspect, the invention provides the use of a PDE5 inhibitor in combination with a 5HT1a agonist in the manufacture of a medicament for the treatment of FSAD and HSDD in a subject suffering from FSAD and concurrent significant HSDD.

The term “significant HSDD” as defined herein means a level of HSDD which causes some degree of personal distress to the female subject. Preferably significant HSDD means a level of HSDD which causes some degree of distress and is measurable.

Preferably the HSDD is measurable through evaluation by a clinician using a semi-structured questionnaire.

More preferably significant HSDD means a level of HSDD which causes some degree of distress and is measurable as a score of less than or equal to 16 on the desire domain in the Sexual Function Questionaire (SFQ) hereinbelow.

A female subject with FSAD and significant HSDD may occasionally experience a slight increase in her desire, for example as a result of psychological factors. It will be appreciated that such a subject typically and generally has significant HSDD and is therefore included within the scope of the invention.

The term “concurrent” as used herein means a subject who experiences FSAD at the same time as experiencing significant HSDD. The term “concurrent” as defined herein does not encompass female subjects with situational HSDD, i.e. subjects who normally experience satisfactory levels of desire and who are normally able to become aroused, but occasionally are unable to experience any satisfactory levels of desire and arousal as a result of external factors, for example partner specific HSDD.

In an embodiment of the invention, the female subject is oestrogen and androgen replete. Replete levels of the oestrogen and androgen may already exist in the subject or they may be achieved artificially. Replete levels or oestrogen may be achieved artificially by administration of estradiol, estrone, estriol, a synthetic oestrogen (for example oestrogen benzoate), an agent which causes the body to produce oestrogen and/or an oestrogen receptor modulator/agonist (for example raloxifene or lasofoxifene). Replete levels of androgen may be achieved by administration of an androgen (such as include androsterone, dehydro-androsterone, testosterone, androstanedione or a synthetic androgen), an agent which causes the body to produce androgen and/or androgen receptor modulator/agonist (for example tibolone)

As used herein the term replete means having concentrations of oestrogen and androgen equal to or greater than minimum physiological concentrations found in a normal subject.

Oestrogen is the general term for any substance having the physiological activity of oestradiol. It includes natural and synthetic oestrogens. Naturally occurring oestrogens include oestradiol, oestrone, oestriol and their conjugates, predominantly protein-bound. An example of a synthetic oestrogen is oestradiol benzoate. In a preferred embodiment the subject has a concentration of oestrogen equal to or greater than 40 picogrammes per millilitre of blood.

Oestradiol concentration provides a reliable measure of total oestrogen levels in the body. The physiological concentration of oestradiol varies depending on the stage of ovulation. The minimum concentration of oestradiol (protein-bound and free) in a normal women is approximately 40 picogrammes per millilitre of blood. Therefore in a preferred embodiment the subject has a concentration of oestradiol (protein-bound and free) equal to or greater than 40 picogrammes per millilitre of blood. Methods and kits for determining oestradiol blood concentrations are well-known to the skilled person, for example Coat-a-Count® Esradiol [available through DPC®(Diagnostic Products Corporation, 5700 West 96th Street, Los Angeles, Calif. 900456-5597] provides a kit to measure the concentration of protein-bound and free oestradiol.

Androgen is the collective term for a group of steroids, both natural and artificial. In females androgens are produced by the ovaries and adrenocortex. The natural androgens include androsterone, dehydroandrosterone, testosterone and androstanedione. Testosterone is by far the most potent natural hormone. Testosterone circulates in the body almost entirely bound to proteins. Normally less than one percent is free. Free testosterone provides an accurate measure of androgen concentration. The free-testosterone concentration in a normal pre-menopausal women is approximately 0.9 picogrammes per millilitre of blood. Therefore in a preferred embodiment the subject has a concentration of free testosterone equal to or greater than 0.9 picogrammes per millilitre of blood. Methods and kits for determining free-testosterone blood concentrations are well-known to the skilled person, for example Coat-a-Count® Esradiol [available through DPC®(Diagnostic Products Corporation, 5700 West 96th Street, Los Angeles, Calif. 900456-5597].

The female subject can be pre-menopausal, peri-menopausal, post-menopausal or surgically menopausal i.e. post-hysterectomy. In a preferred embodiment the subject is post-menopausal.

The skilled person will appreciate that the subject in addition to suffering from FSAD may also suffer from FOD or sexual pain disorders provided these are secondary to FSAD.

Hereinafter the term “the PDE5 inhibitor” means the PDE5 inhibitors for use with the invention. The term includes pharmaceutically acceptable salts, solvates and polymorphs of the PDE5 inhibitors for use with the invention.

The suitability of the PDE5 inhibitor can be readily determined by evaluation of its potency and selectivity using literature methods followed by evaluation of its toxicity, absorption, metabolism, pharmacokinetics, etc in accordance with standard pharmaceutical practice.

Preferably, the PDE5 inhibitors have an IC50 against the PDE5 enzyme of less than 100 nanomolar, more preferably, at less than 50 nanomolar.

IC50 values for the PDE5 inhibitors may be determined using the PDE5 assay in the Test Methods Section hereinafter.

Preferably the PDE5 inhibitors are selective for the PDE5 enzyme. Preferably they have a selectivity of PDE5 over PDE3 of greater than 100 more preferably greater than 300. More preferably the PDE5 has a selectivity over both PDE3 and PDE4 of greater than 100, more preferably greater than 300.

Selectivity ratios may readily be determined by the skilled person, by ratio of corresponding IC50 values for the particular enzymes concerned. IC50 values for the PDE3 and PDE4 enzyme may be determined using established literature methodology, see S A Ballard et al, Journal of Urology, 1998, vol.159, pages 2164-2171.

Preferably the PDE5 inhibitors have an IC50 against PDE5 of less than 100 nM and a selectivity over PDE3 of greater than 100 fold.

Examples of PDE5 inhibitors for use with the invention are:

The pyrazolo[4,3-d]pyrimidin-7-ones disclosed in EP-A-0463756; the pyrazolo[4,3-d]pyrimidin-7-ones disclosed in EP-A-0526004; the pyrazolo [4,3-d]pyrimidin-7-ones disclosed in published international patent application WO 93/06104; the isomeric pyrazolo[3,4-d]pyrimidin-4-ones disclosed in published international patent application WO 93/07149; the quinazolin-4-ones disclosed in published international patent application WO 93/12095; the pyrido[3,2-d]pyrimidin-4-ones disclosed in published international patent application WO 94/05661; the purin-6-ones disclosed in published international patent application WO 94/00453; the pyrazolo[4,3-d]pyrimidin-7-ones disclosed in published international patent application WO 98/49166; the pyrazolo[4,3-d]pyrimidin-7-ones disclosed in published international patent application WO 99/54333; the pyrazolo[4,3-d]pyrimidin-4-ones disclosed in EP-A-0995751; the pyrazolo[4,3-d]pyrimidin-7-ones disclosed in published international patent application WO 00/24745; the pyrazolo[4,3-d]pyrimidin-4-ones disclosed in EP-A-0995750; the compounds disclosed in published international application WO95/19978; the compounds disclosed in published international application WO 99/24433 and the compounds disclosed in published international application WO 93/07124.

The pyrazolo[4,3-d]pyrimidin-7-ones disclosed in published international application WO 01/27112; the pyrazolo[4,3-d]pyrimidin-7-ones disclosed in published international application WO 01/27113; the compounds disclosed in EP-A-1092718 and the compounds disclosed in EP-A-1092719.

Preferred PDE5 inhibitors for use with the invention:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine (see EP-A-0463756);
5-(2-ethoxy-5-morpholinoacetylphenyl)-1-methyl-3-n-propyl-1,6-dihydro-7H-pyrazolo[4,3-d]pyrimidin-7-one (see EP-A-0526004);
3-ethyl-5-[5-(4-ethylpiperazin-1-ylsulphonyl)-2-n-propoxyphenyl]-2-(pyridin-2-yl)methyl-2,6-dihydro-7H-pyrazolo[4,3-d]pyrimidin-7-one (see WO98/49166);
3-ethyl-5-[5-(4-ethylpiperazin-1-ylsulphonyl)-2-(2-methoxyethoxy)pyridin-3-yl]-2-(pyridin-2-yl)methyl-2,6-dihydro-7H-pyrazolo[4,3-d]pyrimidin-7-one (see WO99/54333);
(+)-3-ethyl-5-[5-(4-ethylpiperazin-1-ylsulphonyl)-2-(2-methoxy-1(R)-methylethoxy)pyridin-3-yl]-2-methyl-2,6-dihydro-7H-pyrazolo[4,3-d]pyrimidin-7-one, also known as 3-ethyl-5-{5-[4-ethylpiperazin-1-ylsulphonyl]-2-([(1R)-2-methoxy-1-methylethyl]oxy)pyridin-3-yl}-2-methyl-2,6-dihydro-7H-pyrazolo[4,3-d]pyrimidin-7-one (see WO99/54333);
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one, also known as 1-{6-ethoxy-5-[3-ethyl-6,7-dihydro-2-(2-methoxyethyl)-7-oxo-2H-pyrazolo[4,3-d]pyrimidin-5-yl]-3-pyridylsulphonyl}-4-ethylpiperazine (see WO 01/27113, Example 8);
5-[2-iso-Butoxy-5-(4-ethylpiperazin-1-ylsulphonyl)pyridin-3-yl]-3-ethyl-2-(1-methylpiperidin-4-yl)-2,6-dihydro-7H-pyrazolo[4,3-d]pyrimidin-7-one (see WO 01/27113, Example 15);
5-[2-Ethoxy-5-(4-ethylpiperazin-1-ylsulphonyl)pyridin-3-yl]-3-ethyl-2-phenyl-2,6-dihydro-7H-pyrazolo[4,3-d]pyrimidin-7-one (see WO 01/27113, Example 66);
5-(5-Acetyl-2-propoxy-3-pyridinyl)-3-ethyl-2-(1-isopropyl-3-azetidinyl)-2,6-dihydro-7H-pyrazolo[4,3-d]pyrimidin-7-one (see WO 01/27112, Example 124);
5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one (see WO 01/27112, Example 132);
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351), i.e. the compound of examples 78 and 95 of published international application WO95/19978, as well as the compound of examples 1, 3, 7 and 8;
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil) also known as 1-[[3-(3,4-dihydro-5-methyl-4-oxo-7-propylimidazo[5,1-f]-as-triazin-2-yl)-4-ethoxyphenyl]sulphonyl]4-ethylpiperazine, i.e. the compound of examples 20, 19, 337 and 336 of published international application WO99/24433; and
the compound of example 11 of published international application WO93/07124 (EISAI); and
compounds 3 and 14 from Rotella D P, J. Med. Chem., 2000, 43, 1257.

Still further PDE5 inhibitors for use with the invention include: 4-bromo-5-(pyridylmethylamino)-6-[3-(4-chlorophenyl)-propoxy]-3(2H)pyridazinone; 1-[4-[(1,3-benzodioxol-5-ylmethyl)amiono]-6-chloro-2-quinozolinyl]-4-piperidine-carboxylic acid, monosodium salt; (+)-cis-5,6a,7,9,9,9a-hexahydro-2-[4-(trifluoromethyl)-phenylmethyl-5-methyl-cyclopent-4,5]imidazo[2,1-b]purin-4(3H)one; furazlocillin; cis-2-hexyl-5-methyl-3,4,5,6a,7,8,9,9a-octahydrocyclopent[4,5]-imidazo[2,1-b]purin-4-one; 3-acetyl-1-(2-chlorobenzyl)-2-propylindole-6-carboxylate; 3-acetyl-1-(2-chlorobenzyl)-2-propylindole-6-carboxylate; 4-bromo-5-(3-pyridylmethylamino)-6-(3-(4-chlorophenyl) propoxy)-3-(2H)pyridazinone; 1-methyl-5(5-morpholinoacetyl-2-n-propoxyphenyl)-3-n-propyl-1,6-dihydro-7H-pyrazolo(4,3-d)pyrimidin-7-one; 1-[4-[(1,3-benzodioxol-5-ylmethyl)amino]-6-chloro-2-quinazolinyl]-4-piperidinecarboxylic acid, monosodium salt; Pharmaprojects No. 4516 (Glaxo Wellcome); Pharmaprojects No. 5051 (Bayer); Pharmaprojects No. 5064 (Kyowa Hakko; see WO 96/26940); Pharmaprojects No. 5069 (Schering Plough); GF-196960 (Glaxo Wellcome); E-8010 and E4010 (Eisai); Bay-38-3045 & 38-9456 (Bayer) and Sch-51866.

The contents of the published patent applications and journal articles and in particular the general formulae of the therapeutically active compounds of the claims and exemplified compounds therein are incorporated herein in their entirety by reference thereto.

More preferred PDE5 inhibitors for use with the invention are selected from the group:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);
(6R,12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351);
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); and
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof.

A particularly preferred PDE5 inhibitor is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) (also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine) and pharmaceutically acceptable salts thereof. Sildenafil citrate is a preferred salt.

Hereinafter the term “the 5HT1a agonist” means the 5HT1a agonists for use with the invention. The term includes pharmaceutically acceptable salts, solvates and polymorphs of the 5HT1a agonists for use with the invention.

The suitability of the 5HT1a agonists can be readily determined by evaluation of its potency and selectivity using literature methods followed by evaluation of its toxicity, absorption, metabolism, pharmacokinetics, etc in accordance with standard pharmaceutical practice.

Preferably, the 5HT1a agonists have an affinity for the recombinant human 5HT1a receptor with a Ki of less than or equal to 300 nM, preferably with a Ki of less than or equal to 100 nM, more preferably with a Ki of less than or equal to 30 nM, yet more preferably with a Ki of less than or equal to 10 nM, most preferably with a Ki of less than or equal to 1 nM.

EC50 values for the 5HT1a agonists may be determined using the 5HT1a assay in the Test Methods Section hereinafter. Preferably the agonists have an EC50 less than or equal to 300 nM, preferably with an EC50 of less than or equal to 100 nM, more preferably with an EC50 of less than or equal to 30 nM, yet more preferably with an EC50 of less than or equal to 10 nM, most preferably with a EC50 of less than or equal to 1 nM.

Preferably the 5HT1a agonists are selective for the 5HT1a receptor over alphaadrenoceptors and dopamine.

Examples of 5HT1a agonists for use with the invention are:
Zaprasidone, buspirone HCl, Urapidil, Tandosporine, Sunepitron, Ebalzotan, Ipsapirone, Zalospirone, Gepirone, Repinotan, Alnespirone, MKC242, Eptapirone, SR57746A, AP-521, SUN-N4057, Lesopitron, DU-125530, VML-670, Flesinoxan, E6265, Flibanserin, buspar, AP-521, SUN-N4057, LY293284, LY301317 and 8-OH-DPAT.

Particularly preferred is Flibanserin

Particularly preferred combinations are Sildenafil or 2-(Methoxyethyl)-5-[2-ethoxy-5-(4-ethylpiperazin-1-ylsulfonyl)pyridin-3-yl]-3-ethyl-2,6-dihydro-7H-pyrazolo[4,3-d]pyrimidin-7-one with Flibanserin.

Oral bioavailablity refers to the proportion of an orally administered drug that reaches the systemic circulation. The factors that determine oral bioavailability of a drug are dissolution, membrane permeability and metabolic stability. Typically, a screening cascade of firstly in vitro and then in vivo techniques is used to determine oral bioavailablity.

Dissolution, the solubilisation of the drug by the aqueous contents of the gastro-intestinal tract (GIT), can be predicted from in vitro solubility experiments conducted at appropriate pH to mimic the GIT. Preferably the PDE5 inhibitors have a minimum solubility of 50 mcg/ml. Solubility can be determined by standard procedures known in the art such as described in Adv. Drug Deliv. Rev. 23, 3-25,1997.

Membrane permeability refers to the passage of a compound through the cells of the GIT. Lipophilicity is a key property in predicting this and is defined by in vitro Log D_{7.4} measurements using organic solvents and buffer. Preferably the PDE5 inhibitors have a Log D_{7.4} of −2 to +4, more preferably −1 to +3. The log D can be determined by standard procedures known in the art such as described in J. Pharm. Pharmacol. 1990, 42:144.

Cell monolayer assays such as CaCo₂ add substantially to prediction of favourable membrane permeability in the presence of efflux transporters such as p-glycoprotein, so-called caco-2 flux. Preferably, the PDE5 inhibitors have a caco-2 flux of greater than 2×10⁻⁶ cms⁻¹, more preferably greater than 5×10⁻⁶ cms⁻¹. The caco flux value can be determined by standard procedures known in the art such as described in J. Pharm. Sci, 1990, 79, 595-600

Metabolic stability addresses the ability of the GIT or the liver to metabolise compounds during the absorption process: the first pass effect. Assay systems such as microsomes, hepatocytes etc are predictive of metabolic liability. Preferably the PDE5 inhibitors show metabolic stability in the assay system that is commensurate with an hepatic extraction of less then 0.5. Examples of assay systems and data manipulation are described in Curr. Opin. Drug Disc. Devel., 201, 4, 36-44, Drug Met. Disp.,2000, 28, 1518-1523

Because of the interplay of the above processes further support that a drug will be orally bioavailable in humans can be gained by in vivo experiments in animals. Absolute bioavailability is determined in these studies by administering the compound separately or in mixtures by the oral route. For absolute determinations (% absorbed) the intravenous route is also employed. Examples of the assessment of oral bioavailability in animals can be found in Drug Met. Disp.,2001, 29, 82-87; J. Med Chem, 1997, 40, 827-829, Drug Met. Disp.,1999, 27, 221-226.

The PDE5 inhibitors and 5HT1a agonists may also be combined with one or more additional active agents for treating sexual dysfunction, particularly FSAD in subjects with concurrent significant HSDD. The additional active agents may be selected from the following list:
1) one or more naturally occurring or synthetic prostaglandins or esters thereof (suitable prostaglandins for use herein include compounds such as alprostadil, prostaglandin E₁, prostaglandin E₀, 13,14-dihydroprosta glandin E₁, prostaglandin E₂, eprostinol, natural synthetic and semi-synthetic prostaglandins and derivatives thereof including those described in WO-00033825 and/or U.S. Pat. No. 6,037,346 issued on 14 Mar. 2000 all incorporated herein by reference, PGE₀, PGE₁, PGA₁, PGB₁, PGF₁α, 19-hydroxy PGA₁, 19-hydroxy -PGB₁, PGE₂, PGB₂, 19-hydroxy-PGA₂, 19-hydroxy-PGB₂, PGE₃α, carboprost tromethamine dinoprost, tromethamine, dinoprostone, lipo prost, gemeprost, metenoprost, sulprostune, tiaprost and moxisylate);
2) one or more α-adrenergic receptor antagonists (also known as α-adrenoceptor blockers, α-receptor blockers or α-blockers); suitable α₁-adrenergic receptor antagonists include: phentolamine, prazosin, phentolamine mesylate, trazodone, alfuzosin, indoramin, naftopidil, tamsulosin, phenoxybenzamine, rauwolfa alkaloids, Recordati 15/2739, SNAP 1069, SNAP 5089, RS17053, SL 89.0591, doxazosin, terazosin and abanoquil; suitable α₂-adrenergic receptor antagonists include dibenarnine, tolazoline, trimazosin, efaroxan, yohimbine, idazoxan clonidine and dibenarnine; suitable non-selective α-adrenergic receptor antagonists include dapiprazole; further α-adrenergic receptor antagonists are described in PCT application WO99/30697 published on 14 Jun. 1998 and U.S. Pat. Nos. 4,188,390; 4,026,894; 3,511,836; 4,315,007; 3,527,761; 3,997,666; 2,503,059; 4,703,063; 3,381,009; 4,252,721 and 2,599,000 each of which is incorporated herein by reference;
3) one or more NO-donor (NO-agonist) compounds (suitable NO-donor compounds for use herein include organic nitrates, such as mono-di or tri-nitrates or organic nitrate esters including glyceryl brinitrate (also known as nitroglycerin), isosorbide 5-mononitrate, isosorbide dinitrate, pentaerythritol tetranitrate, erythrityl tetranitrate, sodium nitroprusside (SNP), 3-morpholinosydnonimine molsidomine, S-nitroso-N-acetyl penicilliamine (SNAP) S-nitroso-N-glutathione (SNO-GLU), N-hydroxy-L-arginine, amylnitrate, linsidomine, linsidomine chlorohydrate, (SIN-1) S-nitroso-N-cysteine, diazenium diolates, (NONOates), 1,5-pentanedinitrate, L-arginene, ginseng, zizphi fructus, molsidomine, Re-2047, nitrosylated maxisylyte derivatives such as NMI-678-11 and NMI-937 as described in published PCT application WO 0012075);
4) one or more potassium channel openers or modulators (suitable potassium channel openers/modulators for use herein include nicorandil, cromokalim, levcromakalim, lemakalim, pinacidil, cliazoxide, minoxidil, charybdotoxin, glyburide, 4-aminopyridine, BaCl₂);
5) one or more dopaminergic agents, preferably apomorphine or a selective D₂, D₃ or D₂/D₃agonist such as, pramipexole and ropirinol (as claimed in WO-0023056), PNU95666 (as claimed in WO-0040226);
6) one or more vasodilator agents (suitable vasodilator agents for use herein include nimodepine, pinacidil, cyclandelate, isoxsuprine, chloroprumazine, halo peridol, Rec 15/2739, trazodone);
7) one or more thromboxane A2 agonists;
8) one or more ergot alkoloids (suitable ergot alkaloids are described in U.S. Pat. No. 6,037,346 issued on 14 Mar. 2000 and include acetergamine, brazergoline, bromerguride, cianergoline, delorgotrile, disulergine, ergonovine maleate, ergotamine tartrate, etisulergine, lergotrile, lysergide, mesulergine, metergoline, metergotamine, nicergoline, pergolide, propisergide, proterguride, terguride);
9) one or more compounds which modulate the action of natruretic factors in particular atrial naturetic factor (also known as atrial naturetic peptide), B type and C type naturetic factors such as inhibitors or neutral endopeptidase (see later);
10) one or more angiotensin receptor antagonists such as losartan;
11) one or more substrates for NO-synthase, such as L-arginine;
12) one or more calcium channel blockers such as amlodipine;
13) one or more antagonists of endothelin receptors and inhibitors or endothelin-converting enzyme;
14) one or more cholesterol lowering agents such as statins (e.g. atorvastatin/Lipitor—trade mark) and fibrates;
15) one or more antiplatelet and antithrombotic agents, e.g. tPA, uPA, warfarin, hirudin and other thrombin inhibitors, heparin, thromboplastin activating factor inhibitors;
16) one or more insulin sensitising agents such as rezulin and hypoglycaemic agents such as glipizide;
17) one or more acetylcholinesterase inhibitors such as donezipil;.
18) one or more estrogen receptor modulators and/or estrogen agonists and/or estrogen antagonists, preferably raloxifene, tibolone or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;
19) one or more further PDE inhibitors, particularly a PDE 2, 7 or 8 inhibitor, preferably a PDE2 inhibitor, said inhibitors preferably having an IC50 against the respective enzyme of less than 100 nM;
20) one or more of an NPY (neuropeptide Y) inhibitor, more particularly NPY1 or NPY5 inhibitor, preferably NPY1 inhibitor, preferably said NPY inhibitors (including NPY Y1 and NPY Y5) having an IC50 of less than 100 nM, more preferably less than 50 nM (an assay for identifying NPY inhibitors is presented in WO-A-98/52890 (see page 96, lines 2 to 28));
21) one or more of vasoactive intestinal protein (VIP), VIP mimetic, VIP analogue, more particularly mediated by one or More of the VIP receptor subtypes VPAC1,VPAC or PACAP (pituitory adenylate cyclase activating peptide), one or more of a VIP receptor agonist or a VIP analogue (eg Ro-125-1553) or a VIP fragment, one or more of a α-adrenoceptor antagonist with VIP combination eg Invicorp, Aviptadil);
22) one or more of a melanocortin receptor agonist or modulator or melanocortin enhancer, such as melanotan II, PT-14, PT-141 or compounds claimed in WO-09964002, WO-00074679, WO-09955679, WO-00105401, WO-00058361, WO-00114879, WO-00113112, WO-09954358;
23) one or more of a serotonin receptor agonist, antagonist or modulator, more particularly agonists, antagonists or modulators for example 5HT2A, 5HT2C, 5HT3, 5HT6 and/or 5HT7 receptors, including those described in WO-09902159, WO-00002550 and/or WO-00028993;
24) one or more of an androgen such as androsterone, dehydroandrosterone, testosterone, androstanedione and a synthetic androgen;
25) one or more of an androgen receptor modulator, for example tibolone;
26) one or more of an oestrogen, such as oestradiol, oestrone, oestriol and a synthetic estrogen, such as oestrogen benzoate);
27) one or more of a modulator of transporters for noradrenaline, dopamine and/or serotonin, such as bupropion, GW-320659;
28) one or more of a purinergic receptor agonist and/or modulator;
29) one or more of a neurokinin (NK) receptor antagonist, including those described in WO-09964008;
30) one or more of an opioid receptor agonist, antagonist or modulator, preferably agonists for the ORL-1 receptor;
31) one or more of an agonist or modulator for oxytocin/vasopressin receptors, preferably a selective oxytocin agonist or modulator;
32) one or more of a modulator of cannabinoid receptors;
33) one or more NEP inhibitors, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719; IC50 values against NEP and ACE may be determined using methods described in published patent application EP1097719-A1, paragraphs [0368] to [0376];
34) one or more compounds which inhibit angiotensin-converting enzyme such as enalapril, and one or more combined inhibitors of angiotensin-converting enzyme and neutral endopeptidase such as omapatrilat;
35) one or more of L-DOPA and carbidopa;
36) one or more COX2 inhibitors;
37) pregabalene or gabapentene;
38) one or more non-steroidal anti-inflammatory agents;
39) one or more angiotensin-converting enzyme (ACE) inhibitors, e.g. quinapril;
40) one or more of a bombesin receptor modulator, preferably selective for the BB1 receptor;
41) one or more antidepressants such as buprion;

If a combination of active agents are administered, then they may be administered simultaneously, separately or sequentially.

Preferably, the PDE5 inhibitors (particularly sildenafil) and the 5HT1a agonist may be combined with one or more active agents selected from the following list:
i) one or more of an androgen such as androsterone, dehydro-androsterone, testosterone, androstanedione and a synthetic androgen;
ii) one or more of an oestrogen, such as oestradiol, oestrone, oestriol and a synthetic estrogen, such as oestrogen benzoate);
iii) one or more NEP inhibitors, preferably wherein said NEP is EC 3.4.24.11 and more preferably wherein said NEP inhibitor is a selective inhibitor for EC 3.4.24.11, more preferably a selective NEP inhibitor is a selective inhibitor for EC 3.4.24.11, which has an IC₅₀ of less than 100 nM (e.g. ompatrilat, sampatrilat) suitable NEP inhibitor compounds are described in EP-A-1097719;
v) one or more of an NPY1 having an IC50 of less than 100 nM, more preferably less than 50 nM, for examples see published European Patent Application EP1 097 718 A1;
vi) one or more estrogen receptor modulators and/or estrogen agonists and/or estrogen antagonists, preferably raloxifene, tibolone or lasofoxifene, (−)-cis-6-phenyl-5-[4-(2-pyrrolidin-1-yl-ethoxy)-phenyl]-5,6,7,8-tetrahydronaphthalene-2-ol and pharmaceutically acceptable salts thereof the preparation of which is detailed in WO 96/21656;
vii) one or more of a melanocortin receptor agonist or modulator or melanocortin enhancer, such as melanotan II, PT-14, PT-141 or compounds claimed in WO-09964002, WO-00074679, WO-09955679, WO-00105401, WO-00058361, WO-00114879, WO-00113112, WO-09954358 (preferably PT-141);
viii) one or more dopaminergic agents, preferably apomorphine or a selective D₂, D₃ or D₂/D₃agonist such as, pramipexole and ropirinol (as claimed in WO-0023056), PNU95666 (as claimed in WO-0040226);
ix) one or more of a bombesin receptor modulators;
x) one or more antidepressants such as buprion; and
xi) one or more 5HT1a modulators (for example VML 670).

Particularly preferred combinations for treating FSAD in subjects with concurrent significant HSDD are:
Sildenalfil, Flibanserin and an androgen;
sildenafil, Flibanserin and an oestrogen;
sildenafil, Flibanserin an androgen and an oestrogen;
sildenafil, Flibanserin and lasofoxifene;
sildenafil, Flibanserin, lasofoxifene and an androgen;
sildenafil, Flibanserin, lasofoxifene and an oestrogen; or
sildenafil, Flibanserin, lasofoxifene, an androgen and an oestrogen;
sildenafil, Flibanserin and a NEP inhibitor;
sildenafil, Flibanserin, a NEP inhibitor and an androgen;
sildenafil, Flibanserin, a NEP inhibitor and an oestrogen; or
sildenafil, Flibanserin, a NEP inhibitor, an androgen and an oestrogen;
sildenafil, Flibanserin and a dopaminergic agent (preferably apomorphine);
sildenafil, Flibanserin, a dopaminergic agent (preferably apomorphine) and an androgen;
sildenafil, Flibanserin, a dopaminergic agent (preferably apomorphine) and an oestrogen;
sildenafil, Flibanserin, a dopaminergic agent (preferably apomorphine), an androgen and an oestrogen;
sildenafil, Flibanserin and a melanocortin enhancer (preferably PT-141);
sildenafil, Flibanserin, a melanocortin enhancer (preferably PT-141) and an androgen;
sildenafil, Flibanserin, a melanocortin enhancer (preferably PT-141) and an oestrogen;
sildenafil, Flibanserin, a melanocortin enhancer (preferably PT-141), an androgen and an oestrogen;
sildenafil, Flibanserin and buprion;
sildenafil, Flibanserin, buprion and an androgen;
sildenafil, Flibanseri, buprion and an oestrogen;
sildenafil, Flibanserin, buprion, an androgen and an oestrogen;

The PDE5 inhibitors with 5HT1a agonists and combinations thereof can be administered alone but will generally be administered in admixture with a suitable pharmaceutical excipient, diluent or carrier selected with regard to the intended route of administration and standard pharmaceutical practice.

For example, the PDE5 inhibitors with 5HT1a agonists and combinations thereof can be administered orally, buccally or sublingually in the form of tablets, capsules, multi-particulates, gels, films, ovules, elixirs, solutions or suspensions, which may contain flavouring or colouring agents, for immediate-, delayed-, modified-, sustained-, pulsed- or controlled-release applications. The PDE5 inhibitors with 5HT1a agonists and combinations thereof may also be administered as fast-dispersing or fast-dissolving dosage forms or in the form of a high energy dispersion or as coated particles. Suitable formulations may be in coated or uncoated form, as desired.

Such solid pharmaceutical compositions, for example, tablets, may contain excipients such as microcrystalline cellulose, lactose, sodium citrate, calcium carbonate, dibasic calcium phosphate, glycine and starch (preferably corn, potato or tapioca starch), disintegrants such as sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

The following formulation examples are illustrative only and are not intended to limit the scope of the invention. Active ingredient means a PDE5 inhibitor with 5HT1a agonists or combination thereof.

Formulation 1:

A tablet is prepared using the following ingredients: Active ingredient (50 mg) is blended with cellulose (microcrystalline), silicon dioxide, stearic acid (fumed) and the mixture is compressed to form tablets.

Formulation 2:

An intravenous formulation may be prepared by combining active ingredient (100 mg) with isotonic saline (1000 ml)

The tablets are manufactured by a standard process, for example, direct compression or a wet or dry granulation process. The tablet cores may be coated with appropriate overcoats.

Solid compositions of a similar type may also be employed as fillers in gelatin or HPMC capsules. Preferred excipients in this regard include lactose, starch, a cellulose, milk sugar or high molecular weight polyethylene glycols. For aqueous suspensions and/or elixirs, the PDE5 inhibitors with 5HT1a agonists may be combined with various sweetening or flavouring agents, colouring matter or dyes, with emulsifying and/or suspending agents and with diluents such as water, ethanol, propylene glycol and glycerin, and combinations thereof.

Modified release and pulsatile release dosage forms may contain excipients such as those detailed for immediate release dosage forms together with additional excipients that act as release rate modifiers, these being coated on and/or included in the body of the device. Release rate modifiers include, but are not exclusively limited to, hydroxypropylmethyl cellulose, methyl cellulose, sodium carboxymethylcellulose, ethyl cellulose, cellulose acetate, polyethylene oxide, Xanthan gum, Carbomer, ammonio methacrylate copolymer, hydrogenated castor oil, carnauba wax, paraffin wax, cellulose acetate phthalate, hydroxypropylmethyl cellulose phthalate, methacrylic acid copolymer and mixtures thereof. Modified release and pulsatile release dosage forms may contain one or a combination of release rate modifying excipients. Release rate modifying excipients may be present both within the dosage form i.e. within the matrix, and/or on the dosage form, i.e. upon the surface or coating.

Fast dispersing or dissolving dosage formulations (FDDFs) may contain the following ingredients: aspartame, acesulfame potassium, citric acid, croscarmellose sodium, crospovidone, diascorbic acid, ethyl acrylate, ethyl cellulose, gelatin, hydroxypropylmethyl cellulose, magnesium stearate, mannitol, methyl methacrylate, mint flavouring, polyethylene glycol, fumed silica, silicon dioxide, sodium starch glycolate, sodium stearyl fumarate, sorbitol, xylitol. The terms dispersing or dissolving as used herein to describe FDDFs are dependent upon the solubility of the drug substance used i.e. where the drug substance is insoluble a fast dispersing dosage form can be prepared and where the drug substance is soluble a fast dissolving dosage form can be prepared.

The PDE5 inhibitors with 5HT1a agonists and combinations thereof can also be administered parenterally, for example, intracavernouslly, intravenously, intra-arterially, intraperitoneally, intrathecally, intraventricularly, intraurethrally, intrasternally, intracranially, intramuscularly or subcutaneously, or they may be administered by infusion or needleless injection techniques. For such parenteral administration they are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The following dosage levels and other dosage levels herein are for the average human subject having a weight range of about 65 to 70 kg. The skilled person will readily be able to determine the dosage levels required for a subject whose weight falls outside this range, such as children and the elderly.

The dosage of the PDE5 inhibitor with 5HT1a agonists in such formulations will depend on its potency, but can be expected to be in the range of from 1 to 500 mg for administration up to three times a day. In the case of sildenafil, a preferred dose is in the range 10 to 100 mg (e.g. 10, 25, 50 and 100 mg) which can be administered once, twice or three times a day (preferably once). However the precise dose will be as determined by the prescribing physician and will depend on the age and weight of the subject and severity of the symptoms.

For oral and parenteral administration to human patients, the daily dosage level of the PDE5 inhibitors will usually be from to 5 to 500 mg/kg (in single or divided doses).

Thus tablets or capsules may contain from 5 mg to 250 mg (for example 10 to 100 mg) of the PDE5 inhibitor for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention. The skilled person will appreciate that the PDE5 inhibitors may be taken as a single dose as needed or desired (i.e. prn). It is to be appreciated that all references herein to treatment include acute treatment (taken as required) and chronic treatment (longer term continuous treatment).

The PDE5 inhibitors with 5HT1a agonists and combinations thereof can also be administered intranasally or by inhalation and are conveniently delivered in the form of a dry powder inhaler or an aerosol spray presentation from a pressurised container, pump, spray, atomiser or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the PDE5 inhibitor and a suitable powder base such as lactose or starch.

Aerosol or dry powder formulations are preferably arranged so that each metered dose or “puff” contains from 1 μg to 50 mg of a PDE5 inhibitor for delivery to the patient. The overall daily dose with an aerosol will be in the range of from 1 μg to 50 mg which may be administered in a single dose or, more usually, in divided doses throughout the day.

Alternatively, the PDE5 inhibitors with 5HT1a agonists and combinations thereof can be administered in the form of a suppository or pessary, or they may be applied topically in the form of a gel, hydrogel, lotion, solution, cream, ointment or dusting powder. The PDE5 inhibitors with 5HT1a agonists and combinations thereof may also be dermally or transdermally administered, for example, by the use of a skin patch, depot or subcutaneous injection. They may also be administered by the pulmonary or rectal routes.

For application topically to the skin, the PDE5 inhibitors with 5HT1a agonists and combinations thereof can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

The PDE5 inhibitors with 5HT1a agonists and combinations thereof may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

Oral administration of the PDE5 inhibitors with 5HT1a agonists and combinations thereof is a preferred route, being the most convenient. In circumstances where the recipient suffers from a swallowing disorder or from impairment of drug absorption after oral administration, the drug may be administered parenterally, sublingually or buccally.

Transdermal administration of the PDE5 inhibitors with 5HT1a agonists and combinations thereof is a further preferred route, particularly local to the female genitalia, preferably intravaginally. A preferred method of transdermal administration of oestrogen and testosterone is using a skin patch, depot or implants.

A preferred dose of an estrogen for combination with the PDE5 inhibitor and 5HT1a agonist is in the range 0 to 5 mg per day.

A preferred dose of an androgen for combination with the PDE5 inhibitor and 5HT1a agonist is in the range 0 to 25 mg per day.

Since the invention has an embodiment that relates to treatment of sexual dysfunction, with a combination of compounds which may be co-administered separately, the invention also relates to combining separate pharmaceutical compositions in kit form. Therefore according to a further aspect, the invention provides a kit comprising: a) a first pharmaceutical composition comprising a PDE5 inhibitor and a pharmaceutically acceptable carrier or diluent; b) a second pharmaceutical composition comprising 5HT1a agonist and a pharmaceutically acceptable carrier or diluent; and a container for the two compositions.

According to a further aspect, since the invention has an embodiment that relates to treatment of FSAD with concomitant HSDD, with a combination of compounds which may be co-administered separately, the invention also relates to combining separate pharmaceutical compositions in kit form. Accordingly, the invention provides a kit comprising: a) a first pharmaceutical composition comprising a PDE5 inhibitor and a pharmaceutically acceptable carrier or diluent; b) a second pharmaceutical composition comprising 5HT1a agonist and a pharmaceutically acceptable carrier or diluent; c) a third pharmaceutical composition comprising an androgen and a pharmaceutically acceptable carrier or diluent; d) a fourth pharmaceutical composition comprising an oestrogen and a pharmaceutically acceptable carrier or diluent and a container for the four compositions. The four compositions are separate components intended for co-administration to a female subject suffering from FSAD, wherein the patient has concentrations of hormone less than the physiological levels found in a normal pre-menopausal woman. By “co-administration”, it is meant that the four components can be taken from the kit and combined for administration together as a composition or as part of the same, unitary dosage form, such as an parenterally or orally administered solution. “Co-administration” also includes administering the components separately (e.g. as tablets or capsules), but as part of the same therapeutic treatment program or regimen. “Separate” administration is the preferred mode of administration. The four components need not be administered at essentially the same time, although they can be if so desired. Thus “co-administration” includes, for example administering all four components as separate dosages or dosage forms and at essentially the same time. “Co-administration” also includes separate administration at different times, in any order, and if preferred by different routes of administration. If administered separately it is preferred that the four components be administered at essentially the same time. If administered separately and at different times, it is preferred that the four components be administered within 24 hours of each other. If administered separately, It is preferred that the four components be administered by the same route. An example of a kit is the so-called blister pack well known in the packaging industry particularly for packaging pharmaceutical dosage forms.

It will be appreciated that the invention covers the following further aspects and that the embodiments specified hereinabove for the first aspect extend to these aspects:
i) a PDE5 inhibitor with a 5HT1a agonist for treating sexual dysfunction;
ii) a PDE5 inhibitor with a 5HT1a agonist for treating FSAD in a subject who has concurrent significant HSDD;
iii) a pharmaceutical combination (for simultaneous, separate or sequential administration) for treating sexual dysfunction comprising a PDE5 inhibitor and a 5HT1a agonist, an oestrogen, an androgen and optionally an additional active agent as hereinabove defined;
iv) a pharmaceutical combination (for simultaneous, separate or sequential administration) for treating FSAD in a subject who has concurrent significant HSDD comprising a PDE5 inhibitor, a 5HT1a agonist, an oestrogen, an androgen and optionally an additional active agent as hereinabove defined;
iv) the use of a pharmaceutical combination for the manufacture of a medicament for treating FSAD in a subject who has concurrent significant HSDD comprising a PDE5 inhibitor, a 5HT1a agonist, an oestrogen, an androgen and optionally an additional active agent as hereinabove defined;
v) a kit for treating FSAD in a subject who does not have concurrent significant HSDD, the kit comprising: a) a first pharmaceutical composition comprising a PDE5 inhibitor; b) a second pharmaceutical composition comprising a 5HT1a agonist; c) a third pharmaceutical composition comprising an androgen; d) a fourth pharmaceutical composition comprising an oestrogen; e) optionally a pharmaceutical composition comprising an additional active agent as hereinabove defined; and f) a container for the compositions;
vi) a method of treating FSAD in a subject who has concurrent significant HSDD comprising treating said patient with an effective amount of a PDE5 inhibitor and a 5HT1a agonist;
vii) a method of treating FSAD in a subject who has concurrent significant HSDD comprising treating said patient with pharmaceutical combination comprising a PDE5 inhibitor, a 5HT1a agonist, an oestrogen, an androgen and optionally an additional active agent as hereinabove defined; and
viii) a method of treating FSAD in a subject who has concurrent significant HSDD comprising the steps of:
   a) measuring the subject's physiological levels of oestrogen and androgen;
   b) if not replete, administering to the subject an oestrogen and/or an androgen until replete levels are achieved; then
   c) administering a PDE5 inhibitor and a 5HT1a agonist.
      Assay

PDE action potency values referred to herein are determined by the following assays.

Preferred PDE compounds suitable for use in accordance with the present invention are potent and selective PDE5 inhibitors. In vitro PDE inhibitory activities against cyclic guanosine 3′,5′-monophosphate (cGMP) and cyclic adenosine 3′,5′-monophosphate (cAMP) phosphodiesterases can be determined by measurement of their IC₅₀ values (the concentration of compound required for 50% inhibition of enzyme activity).

The required PDE enzymes can be isolated from a variety of sources, including human corpus cavernosum, human and rabbit platelets, human cardiac ventricle, human skeletal muscle and bovine retina, essentially by the method of W. J. Thompson and M. M. Appleman (Biochem., 1971, 10, 311). In particular, the cGMP-specific PDE (PDE5) and the cGMP-inhibited cAMP PDE (PDE3) can be obtained from human corpus cavernosum tissue, human platelets or rabbit platelets; the cGMP-stimulated PDE (PDE2) was obtained from human corpus cavernosum; the calcium/calmodulin (Ca/CAM)-dependent PDE (PDE1) from human cardiac ventricle; the cAMP-specific PDE (PDE4) from human skeletal muscle; and the photoreceptor PDE (PDE6) from bovine retina. Phosphodiesterases 7-11 can be generated from full length human recombinant clones transfected into SF9 cells.

Assays can be performed either using a modification of the “batch” method of W. J. Thompson et al. (Biochem., 1979, 18, 5228) or using a scintillation proximity assay for the direct detection of AMP/GMP using a modification of the protocol described by Amersham plc under product code TRKQ7090/7100. In summary, the effect of PDE inhibitors was investigated by assaying a fixed amount of enzyme in the presence of varying inhibitor concentrations and low substrate, (cGMP or cAMP in a 3:1 ratio unlabelled to [3H]-labeled at a conc ˜⅓ Km) such that IC₅₀≅Kᵢ. The final assay volume was made up to 100 μl with assay buffer [20 mM Tris-HCl pH 7.4, 5 mM MgCl₂, 1 mg/ml bovine serum albumin]. Reactions were initiated with enzyme, incubated for 30-60 min at 30° C. to give <30% substrate turnover and terminated with 50 μl yttrium silicate SPA beads (containing 3 mM of the respective unlabelled cyclic nucleotide for PDEs 9 and 11). Plates were re-sealed and shaken for 20 min, after which the beads were allowed to settle for 30 min in the dark and then counted on a TopCount plate reader (Packard, Meriden, Conn.) Radioactivity units were converted to % activity of an uninhibited control (100%), plotted against inhibitor concentration and inhibitor IC₅₀ values obtained using the ‘Fit Curve’ Microsoft Excel extension.

Suitable assays to identify 5HT1a agonists are disclosed in J Pharmacol Exp Ther. 1998, 284(3), 1082-1094 and in J Pharmacol Toxicol Methods 1998, 40(1), 47-55. Other suitable assays are well known to the man skilled in the art.

Diagnosis of FSAD with concomitant HSDD may be achieved through the use of the Sexual History Interview (“SHI”), administered by sexual health experts for the FSD diagnosis of potential subjects. The SHI was developed by a team of internal clinical personnel at Pfizer and external sexual health experts. The SHI addresses two main aspects relevant to subject inclusion/exclusion; identification of the sub-component of FSD and psychosexual eligibility. These two aspects are key to ensuring appropriate identification of the target population and that the main inclusion and exclusion criteria relevant to psychosexual eligibility are met. The SHI is detailed hereinafter.

### Pfizer Sexual Health Interview (SHI)

A Screening Sexual History Semi-Structured Interview

Administration Guidelines

Time: Schedule at least 90 minutes to complete this process: 60-90 minutes for the Interview; 15-30 minutes to write the justification.

Interviewer: Only a Pfizer-approved FSD expert is authorized to conduct the interview: the function cannot be delegated. The FSD expert is invested with sign-off authority on psychological & sexual history-related protocol inclusion/exclusion criteria: co-signature of both the FSD expert and the principal investigator is required for any subject to enter the study.

Conduct in a private room alone with the candidate (no partners).

The order of questions may be altered at your discretion & questions may be skipped if, in your opinion, sufficient information has been acquired and annotated from preceding questions to satisfy the inclusion/exclusion criteria which apply to that issue. Additional questions are permitted, however, please clearly annotate both the question & the answer.

Annotations must be legible to outside reviewers. Text must be comprehensible, but sentence structure & grammar need not be perfect. A telegraphic writing style is acceptable.

Please read these instructions to the subjects prior to starting the interview:
“I am going to ask you a series of questions, which I would like you to answer as honestly and candidly as you can. If you can, please use a time frame of the last 6 months, however it is okay to bring in other times. If you are unsure, or are uncomfortable, about answering any question, it is perfectly OK to say so.”

In answering these questions, the following definitions apply:
Sexual Activity: Includes any activity which may result in sexual stimulation or sexual pleasure, e.g. intercourse, caressing, foreplay, masturbation (i.e. self-masturbation or your partner masturbating you) and oral sex (i.e. your partner giving you oral sex).
Sexual Intercourse: Sexual activity which results in penetration of your vagina by your partner.
Sexual Stimulation: Caressing, foreplay
Self-stimulation (e.g. masturbation): Caressing or fondling of genitals yourself, your partner caressing them, or by using a device.”

For the first question, you may wish to review the range of typical physical & cognitive changes a woman might experience when sexually stimulated or sexually aroused, including: increased heart rate & breathing; feelings of closeness & intimacy with the partner; nipples becoming more sensitive & erect; increased sensitivity to touch; increased genital sensitivity; pleasurable sensations in the clitoris (pulsating/tingling); sensations of genital warmth & lubrication/wetness; feeling ‘excited’/aroused wanting sexual stimulation to continue.

Question 1 can EITHER be treated as open-ended, with you checking all options volunteered & prompting for items not mentioned OR subject can fill in directly, then you review answers together.

[image]

[image]

[image]

[image]

[image]

[image]

[image]

[image]

[image]

[image]

[image]

[image]

[image]

An additional tool is the use of a female sexual function questionnaire (SFQ) developed by the applicants as published in “Development of a Sexual Function Questionnaire for Clinical Trials of Sexual Function”; as referred to in Heiman, International Journal of Fertility and Women's Health; 2000, 45; 200 (incorporated herein by reference in its entirety) and as detailed hereinafter.

### The Female Sexual Function Questionnaire (SFQ)

Background and Scoring

The Female Sexual Function Questionnaire (SFQ) is a self-report outcomes measure of female sexual function that has been developed to be multi-dimensional and subject-centered.

The SFQ addresses all aspects of the sexual response cycle (desire, arousal, orgasm) as well as pain, which is in keeping with the DSM-IV diagnostic criteria and the newly generated AFUD definitions¹.

The item content of the SFQ was generated from the aggregated responses of 82 women to a semi-structured interview. The content of the interview addressed, amongst other things, women's understanding of the terms commonly used to describe the phases of sexual response (e.g. desire and arousal) and the language that they themselves used to describe these changes. These interviews also addressed some of the consequences of female sexual dysfunction (FSD) for the woman, her partner and their relationship and some of these core issues are also represented within the SFQ item content.

Both the physical and the cognitive aspects of sexual response are evaluated within the SFQ items as these two elements were strongly identified as being important, both in relation to the impact of FSD and to changes in function, both positive and negative, in the interview sample of women. The item content of the SFQ has also been judged to be clinically relevant by an external panel of clinicians with expertise in Psychology, Physiology, Gynaecology, Physical Medicine and the treatment of FSD.

Subsequent use of the SFQ in clinical trials in a large sample of women (approx. 900) has demonstrated that it has excellent psychometric properties and has demonstrated discriminative and construct validity, test-retest reliablility, internal consistency and sensitivity to change. This is the case at both the item level and the domain level (seven domains have been identified through factor analysis: Desire, Arousal (sensation), Arousal (lubrication), Orgasm, Pain, Enjoyment and Partner).

The validity of the SFQ at both the item level and the domain level supports the use of individual SFQ domains as primary endpoints (e.g. Arousal or Orgasm) with the remaining domains or individual items being utilised as secondary endpoints. This approach also ensures that all aspects of sexual function are evaluated in a therapeutic area in which the effects of dysfunction and intervention are not currently wholly understood.

The SFQ has been developed and validated in a number of languages (18) as well as for use in the USA and Australia.

The development of the SFQ has been presented at the Washington Consensus Conference (Bethesda 1998), the Cape Cod FSD Conference (1998) and the International Conference for Medical Studies in Female Health (San Francisco 1999). A manuscript, which briefly describes the SFQ development, has been submitted to the Journal of Women's Health and Gender-Based Medicine (‘Development of a Sexual Function Questionnaire for Clinical Trials of Female Sexual Dysfunction'. F. H Quirk et al.)

An abstract has been published in the International Journal of Fertility and Women's Health².

References:

1. ‘Report of the International Consensus Development Conference on Female Sexual Dysfunction: Definitions and Classifications’, R. Basson et al, Journal of Urology, Vol163, p888-893, 2000.
2. ‘Development of a Sexual Function Questionnaire for Clinical Trials of Female Sexual Dysfunction’, J. Heiman, International Journal of Fertility and Women's Medicine, vol 45, 2, p200, 2000.

SFQ Scoring System (Items, Total, Domains)

Individual Items

The SFQ contains 34 items and each item has between 5 or 7 possible response options.

Items 1-5, 27-28, and 33-34 are scored 1-5 (in ascending order) e.g.

1. Over the last 4 weeks, how often have you had pleasurable thoughts and feelings about sexual activity?

| | |
|---|---|
| Not at all | (1) |
| Rarely | (2) |
| Sometimes | (3) |
| Often | (4) |
| Very often | (5) |

Items 6-14, 16, 20-21, 23-26 are scored 1-5 (in ascending order) with the ‘not applicable’ category (e.g. ‘I did not take part in sexual activity’, I did not have any orgasms') set to ‘missing’. e.g.

6. Over the last 4 weeks, in general, how enjoyable has it been to be sensually touched and caressed by your partner?

I have not been touched or caressed (missing)

| | |
|---|---|
| Not enjoyable | (1) |
| Slightly enjoyable | (2) |
| Moderately enjoyable | (3) |
| Very enjoyable | (4) |
| Extremely enjoyable | (5) |

Items 15 and 19 are scored 0-6 (in ascending order) e.g.

15. Over the last 4 weeks, how often did you take part in sexual activity with penetration (e.g. vaginal penetration and intercourse)?

| | |
|---|---|
| I did not take part in sexual activity | (0) |
| Once/twice | (1) |
| 3-4 times | (2) |
| 5-8 times | (3) |
| 9-12 times | (4) |
| 13-16 times | (5) |
| >16 times | (6) |

Items 17*-18*, 29*, 30 and 31 are scored 1-5 (in descending order) [* For items 17, 18 and 29, the ‘I did not take part in sexual activity’ category is set to ‘missing’.]e.g.

30. Thinking about the last 4 weeks, how much did you worry that your partner may look for another sexual relationship because of problems with your sexual life?

| | |
|---|---|
| Not at all | (5) |
| Slightly | (4) |
| Moderately | (3) |
| Very | (2) |
| Extremely | (1) |

Item 22 is scored from 5-1 with the ‘I did not take part in sexual activity’ scored as ‘missing’ and the ‘I did not take part in sexual activity because of being worried or anxious about pain’ scored as 0 i.e.

22. Over the last 4 weeks, how often have you been worried or anxious about pain during sexual activity?

| I did not take part in sexual activity | (missing) |
|---|---|
| I did not take part in sexual activity because | (0) |
| of being worried or anxious about pain’ | |
| Not at all | (5) |
| Sometimes | (4) |
| Often | (3) |
| Very often | (2) |
| Every time | (1) |

Note: Item 32 is not included in the overall scoring but may be tabulated if desired.
Total Score

A total score may be derived from summing the individual item score for each item, except item 32. The total score range is 30-167.

A higher score indicates better sexual function.

Domain Scores

Seven domains have been identified through factor analysis.

| | | | | *Scores |
|---|---|---|---|---|
| | | | | suggesting |
| | | | | normal |
| Domain | # of items | Items | Score range | function |
| Desire | 6 | 1-4, 15, 28 | 5-31 | >23 |
| Arousal (S) | 4 | 7-10 | 4-20 | >14 |
| Arousal (L) | 2 | 11-12 | 2-10 | >8 |
| Orgasm | 3 | 24-26 | 3-15 | >12 |
| Pain | 3 | 17, 18, 22 | 2-15 | >12 |
| Enjoyment | 6 | 6, 16, 20, 21, | 6-30 | >23 |
| | | 23, 27 | | |
| Partner | 2 | 30, 31 | 2-10 | >8 |

*These scores indicating a high likelihood of normal function have been derived using discriminant analyses from the current database and should be used as guidelines only. There is a band of score below these where functional status (excluding Partner domain) would be considered as borderline depending on other clinical indices. See Table 1.

TABLE 1
SFQ Score ranges indicative of likelihood of sexual dysfunction

| | | | Score range |
|---|---|---|---|
| | | Score range | indicating high |
| | Score range | indicating | probability of |
| | indicating high | borderline sexual | normal sexual |
| Domain | probability of FSD | function | function |
| Desire | 5-17 | 18-22 | 23-31 |
| Arousal (S) | 4-10 | 11-13 | 14-20 |
| Arousal (L) | 2-5 | 6-7 | 8-10 |
| Orgasm | 3-8 | 9-11 | 12-15 |
| Pain | 2-8 | 9-11 | 12-15 |
| Enjoyment | 6-16 | 17-22 | 23-30 |

When used in conjunction with a clinical sexual history interview the SFQ scores should be supportive of information derived from the subject (i.e. if the subject proposes that orgasm is her greatest sexual complaint a score within the range of 3-11 would be expected. A score greater than 12 should prompt a review and further discussion).

Where discrepancies between the SFQ score and the sexual problem (s) derived from the sexual history interview arise the opportunity should be taken to discuss this further with the subject and determine the cause(s) for any discrepancy.

The actual SFQ completed by the subject is as follows:

Sexual Function Questionnaire

These questions ask about your sexual activity over the last 4 weeks. Please answer every question by marking one box with a cross. If you are unsure about how to answer, please give the best answer you can. In answering these questions the following definitions apply:

Sexual Activity includes any activity which may result in sexual stimulation or sexual pleasure e.g. intercourse, caressing, foreplay, masturbation (i.e. self masturbation or your partner masturbating you) and oral sex (i.e. your partner giving you oral sex).

Sexual Life includes both the physical sexual activities and the emotional sexual relationship that you have with your partner.
1. Over the last 4 weeks, how often have you had pleasurable thoughts and feelings about sexual activity? Answer selection: Not at all, Rarely; Sometimes; Often; Very often
2. Over the last 4 weeks, how often have you wanted to be sensually touched and caressed by your partner? Answer selection: Not at all; Rarely; Sometimes; Often; Very often
3 . Over the last 4 weeks, how often have you wanted to take part in sexual activity? Answer selection: Not at all; Rarely; Sometimes; Often; Very often
4 . Over the last 4 weeks, how often have you initiated sexual activity with your partner? Answer selection: Not at all; Rarely; Sometimes; Often; Very often
5. Over the last 4 weeks, how often have you been sensually touched and caressed by your partner? Answer selection: Not at all; Rarely; Sometimes; Often; Very often
6. Over the last 4 weeks, in general, how enjoyable has it been to be sensually touched and caressed by your partner? Answer selection: I have not been touched or caressed; Not enjoyable; Slightly enjoyable; Moderately enjoyable; Very enjoyable; Extremely enjoyable.
7. Over the last 4 weeks, how often did you have a feeling of ‘warmth’ in your vagina/genital area when you took part in sexual activity? Answer selection: I did not take part in sexual activity; Not at all; Sometimes; Often; Very often; Every time
8. Over the last 4 weeks, in general, how much ‘warmth’ did you feel in your vagina/genital area when you took part in sexual activity? Answer selection: I did not take part in sexual activity; None; Slightly ‘warm’; Moderately ‘warm’; Very ‘warm’; Extremely ‘warm’
9. Over the last 4 weeks, how often did you have a sensation of ‘pulsating’ (‘tingling’) in your vagina/genital area when you took part in sexual activity? Answer selection: I did not take part in sexual activity, Not at all; Sometimes; Often; Very often; Every time
10. Over the last 4 weeks, in general, how much ‘pulsating’ (‘tingling’) in your vagina/genital area did you notice when you took part in sexual activity? Answer selection: I did not take part in sexual activity; No sensation; A mild sensation; A moderate sensation; A strong sensation; A very strong sensation
11. Over the last 4 weeks, how often did you notice vaginal wetness/lubrication when you took part in sexual activity? Answer selection: I did not take part in sexual activity; Not at all; Sometimes; Often; Very often; Every time
12. Over the last 4 weeks, in general, how much vaginal wetness/lubrication did you notice when you took part in sexual activity? Answer selection: I did not take part in sexual activity; No wetness/lubrication; Slightly wet/lubricated; Moderately wet/lubricated; Very wet/lubricated; Extremely wet/lubricated
13. Over the last 4 weeks, how often did you have feelings of emotional sexual arousal when you took part in sexual activity? (e.g. feeling excited, feeling ‘turned on’, wanting sexual activity to continue). Answer selection: I did not take part in sexual activity; Not at all; Sometimes; Often; Very often; Every time
14. Over the last 4 weeks, how much emotional sexual arousal did you notice when you took part in sexual activity? (e.g. feeling excited, feeling ‘turned on’, wanting sexual activity to continue). Answer selection: I did not take part in sexual activity; None; Slightly aroused; Moderately aroused; Very aroused; Extremely aroused
15. Over the last 4 weeks, how often did you take part in sexual activity with penetration (e.g. vaginal penetration and intercourse)? Answer selection: I did not take part in sexual activity; Once/twice; 3-4 times; 5-8 times; 9-12 times; 13-16 times; >16 times
16. Over the last 4 weeks, in general, how much did you enjoy penetration and intercourse? Answer selection: I did not take part in sexual activity; Not enjoyable; Slightly enjoyable; Moderately enjoyable; Very enjoyable; Extremely enjoyable
17. Over the last 4 weeks, how often did you experience pain in your vagina/genital area during or after sexual activity (e.g. penetration, intercourse)? Answer selection: I did not take part in sexual activity; Not at all; Sometimes; Often; Very often; Every time
18. Over the last 4 weeks, in general, how much pain did you experience in your vagina/genital area during or after sexual activity (e.g. penetration, intercourse)? Answer selection: I did not take part in sexual activity; No pain; Slightly painful; Moderately painful; Very painful; Extremely painful
19. Over the last 4 weeks, how often did you take part in sexual activity without penetration (e.g. masturbation and oral sex)? Answer selection: I did not take part in sexual activity; Once/twice; 3-4 times; 5-8 times; 9-12 times; 13-16 times; >16 times
20. Over the last 4 weeks, in general, how much did you enjoy sexual activity without penetration (e.g. masturbation, oral sex)? Answer selection: I did not take part in sexual activity; No enjoyment, Slightly enjoyable; Moderately enjoyable; Very enjoyable; Extremely enjoyable
21. Over the last 4 weeks, how often did you feel emotionally close to your partner when you took part in sexual activity? Answer selection: I did not take part in sexual activity; Not at all; Sometimes; Often; Very often; Every time
22. Over the last 4 weeks, how often have you been worried or anxious about pain during sexual activity? Answer selection: I did not take part in sexual activity because of being worried or anxious about pain; Not at all; Sometimes; Often; Very often; Every time
23. Over the last 4 weeks, did you feel good about yourself when you were sexually active? Answer selection: I did not take part in sexual activity; Not at all; Slightly; Moderately; Very; Extremely
24. Over the last 4 weeks, how often did you have an orgasm when you took part in sexual activity (may be with or without a partner)? Answer selection: I did not take part in sexual activity; Not at all; Sometimes; Often; Very often; Every time
25. Over the last 4 weeks, in general, how pleasurable were the orgasms that you had? Answer selection: I did not have any orgasms; Not pleasurable; Slightly pleasurable; Moderately pleasurable; Very pleasurable; Extremely pleasurable
26. Over the last 4 weeks, in general, how easy was it for you to reach orgasm? Answer selection: I did not have any orgasms; Very difficult, Quite difficult, Neither easy nor difficult, Quite easy, Very easy
27. Over the last 4 weeks, how confident have you felt about yourself as a sexual partner? Answer selection: Not at all; Slightly; Moderately; Very; Extremely
28. Thinking about your sexual life over the last 4 weeks, how often did you look forward to sexual activity? Answer selection: Not at all; Rarely; Sometimes; Often; Very often
29. Thinking about your sexual life over the last 4 weeks, did you feel disappointed with your sexual response (e.g. ability to become aroused, lubrication)? Answer selection: I did not take part in sexual acvity; Not at all; Slightly; Moderately; Very; Extremely
30. Thinking about the last 4 weeks, how much did you worry that your partner may look for another sexual relationship because of problems with your sexual life? Answer selection: Not at all; Slightly; Moderately; Very, Extremely
31. Thinking about the last 4 weeks, how much did you worry about your partner's negative feelings about your sexual life (e.g. partner feeling angry, hurt, rejected)? Answer selection: Not at all; Slightly; Moderately; Very; Extremely
32. Thinking about your sexual life over the last 4 weeks, how did you feel about the frequency of your sexual activity? Answer selection: A lot less than you desired; A little less than you desired; About right for you; A little more than you desired; A lot more than you desired
33. In general, how important is being able to have an enjoyable sexual life to you? Answer selection: Not at all; Slightly; Moderately; Very; Extremely
34. Over the last 4 weeks, taking the whole of your sexual life into account, how satisfied have you been? Answer selection: Not satisfied; Slightly satisfied; Moderately satisfied; Very satisfied; Extremely satisfied

1. The use of a PDE5 inhibitor in combination with a 5HT1a agonist in the manufacture of a medicament for the treatment of sexual dysfunction

2. The use as claimed in claim 1 wherein the dysfunction is selected from FSD, FSAD, HSDD, FOD, and MED.

3. The use of a PDE5 inhibitor in combination with a 5HT1a agonist in the manufacture of a medicament for the treatment of FSAD and HSDD in a subject suffering from FSAD and concurrent significant HSDD.

4. The use of a PDE5 inhibitor in combination with a 5HT1a agonist in the preparation of a medicament for the treatment of FSAD in a subject who has concurrent significant HSDD.

5. The use as claimed in claims 1 to 4 wherein the PDE5 inhibitor is selected from the group:
5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil);
(6R, 12aR)-2,3,6,7,12,12a-hexahydro-2-methyl-6-(3,4-methylenedioxyphenyl)-pyrazino[2′,1′:6,1]pyrido[3,4-b]indole-1,4-dione (IC-351);
2-[2-ethoxy-5-(4-ethyl-piperazin-1-yl-1-sulphonyl)-phenyl]-5-methyl-7-propyl-3H-imidazo[5,1-f][1,2,4]triazin-4-one (vardenafil); and
5-[2-ethoxy-5-(4-ethylpiperazin-1-ylsulphonyl)pyridin-3-yl]-3-ethyl-2-[2-methoxyethyl]-2,6-dihydro-7H-pyrazolo[4,3-d]pyrimidin-7-one or 5-(5-Acetyl-2-butoxy-3-pyridinyl)-3-ethyl-2-(1-ethyl-3-azetidinyl)-2,6-dihydro-7H-pyrazolo[4,3-d]pyrimidin-7-one and pharmaceutically acceptable salts thereof.

6. The use as claimed in claims 1 to 4 wherein the PDE5 inhibitor is 5-[2-ethoxy-5-(4-methyl-1-piperazinylsulphonyl)phenyl]-1-methyl-3-n-propyl-1,6-dihydro-7H-pyrazolo[4,3-d]pyrimidin-7-one (sildenafil) (also known as 1-[[3-(6,7-dihydro-1-methyl-7-oxo-3-propyl-1H-pyrazolo[4,3-d]pyrimidin-5-yl)-4-ethoxyphenyl]sulphonyl]-4-methylpiperazine) and pharmaceutically acceptable salts thereof.

7. The use as claimed in claims 1 to 4 wherein the PDE5 inhibitor is Sildenafil citrate.

8. The use as claimed in claims 1 to 4 wherein the 5HT1a agonist is selective for the 5HT1a receptor over alpha-adrenoceptors and dopamine.

9. The use as claimed in claims 1 to 4 wherein the 5HT1a agonist is selected from:
Zaprasidone, buspirone HCl, Urapidil, Tandosporine, Sunepitron, Ebalzotan, Ipsapirone, Zalospirone, Gepirone, Repinotan, Alnespirone, MKC242, Eptapirone, SR57746A, AP-521, SUN-N4057, Lesopitron, DU-125530, VML-670, Flesinoxan, E6265, Flibanserin, buspar, AP-521, SUN-N4057, LY293284, LY301317 and 8-OH-DPAT.

10. The use as claimed in claims 1 to 4 wherein the 5HT1a agonist is Flibanserin

11. A pharmaceutical composition including a PDE5 inhibitor, a 5HT1a agonist and a pharmaceutically acceptable excipient, diluent or carrier.

12. A kit comprising:
a) a first pharmaceutical composition comprising a PDE5 inhibitor and a pharmaceutically acceptable carrier or diluent;
b) a second pharmaceutical composition comprising 5HT1a agonist and a pharmaceutically acceptable carrier or diluent;
and a container for the two compositions.
